(19) United States
(12) Patent Application Publication
(10) Pub. No.: US 2009/0226476 A1
(43) Pub. Date: Sep. 10, 2009

(54) Human Glycoform

(76) Inventor: Marian L. Kruzel, Houston, TX (US)

Correspondence Address: CONLEY ROSE, P.C.;David A. Rose, P. O. BOX 3267, HOUSTON, TX, 77253-3267, US

(21) Appl. No.: 12/398,620

(22) Filed: Mar. 5, 2009

Related U.S. Application Data
(60) Provisional application No. 61/068,713, filed on Mar. 10, 2008.

(51) Int. Cl. A61K 39/00; A61P 37/04

(52) U.S. Cl. 424/185.1

(57) ABSTRACT

The present invention relates to a composition of matter consisting of fully humanized recombinant human lactoferrin (rhLF) that is capable to elicit the immune responses with respect to disease or drug-induced immune impairment in humans.

### CROSS-REFERENCE TO RELATED APPLICATIONS

This application is based on provisional application U.S. Ser. No. 61/068,713 filed Mar. 10, 2008, entitled “Humanized Lactoferrin”, which is incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. 1 R41 A151050-01A1 and 2 R42 A1051050-02, awarded by the National Institutes of Health (National Institute of Allergy and Infectious Diseases).

### FIELD OF INVENTION

The present invention relates to a composition of matter consisting of recombinant human lactoferrin (rhLF) wherein the human DNA clone-expressed lactoferrin is glycosylated and sialylated to have a uniform N-glycan structure (Sia₂Gal₂GlcNAc₂Man₃GlcNAc₂). A specific aspect of the invention is that the human recombinant lactoferrin of the present invention is being glycosylated such as to be the same as endogenous human granulocytic lactoferrin, hence making lactoferrin suitable for systemic (intravenous) administration in humans.

### BACKGROUND OF THE INVENTION

Traditional production of therapeutic glycoproteins relies on mammalian cell culture technology. Glycoproteins produced by mammalian cells invariably display N-glycan heterogeneity resulting in a mixture of glycoforms the composition of which varies from production batch to production batch. The current CHO cell-based manufacturing systems produce proteins with far less uniformity of glycosylation compared to some new generation yeast based systems. Using other alternative technologies for glycoprotein production such as insect cells, transgenic plants, and wild-type fungal systems, N-glycosylation is non-human and the glycoproteins produced in these systems are therefore expected to be immunogenic (Altmann, F., E. Staudacher, I. B. H. Wilson, and L. Marz, Insect cells as hosts for the expression of recombinant glycoproteins. Glycoconjugate Journal, 1999. 16(2): p. 109-123.; Bardor, M., C. Faveeuw, A. C. Fitchette, D. Gilbert, L. Galas, F. Trottein, L. Faye, and P. Lerouge, Immunoreactivity in mammals of two typical plant glyco-epitopes, core alpha(1,3)-fucose and core xylose. Glycobiology, 2003. 13(6): 427-34). Moreover, the extent and type of N-glycosylation has a profound impact on the therapeutic properties of many commercially relevant therapeutic proteins making control of N-glycosylation an emerging field of high importance. Compared to the mammalian cell culture technology the methylotrophic yeast Pichia pastoris has the capability of yielding up to 20 times more product. However, glycoproteins derived from Pichia pastoris as well as other fungal and yeast expression systems display fungal-type high mannose N-linked glycans. These glycans are believed to contribute to reduce the half-life of the glycoprotein in vivo and may be immunogenic, thus limiting the potential therapeutic value of fungal-derived glycoproteins. It has been demonstrated that glycosylation of therapeutic antibodies can have a critical effect on biological activity and pharmacokinetics. The removal of the N-linked oligosaccharide from the CH2 heavy chain region of the antibody abrogates target cell killing by complement-mediated lysis (CML) and antibody-dependent cell-mediated cytotoxicity (ADCC) (Nose M. and Wigzell H. Biological significance of carbohydrate chains on monoclonal antibodies. Proc Natl Acad Sci USA. 1983;80(21):6632-6). Therefore, it was concluded that proper glycosylation of therapeutic proteins, compatible with the host counterparts, is important in the clinical outcome of therapy.

Human N-glycosylation is a multi-step process localized to the secretory pathway of cells. The complex metabolic engineering endeavor of replicating the mammalian glycosylation machinery in yeast requires the cloning and functional expression of a large number of foreign glycosylation pathway enzymes in the host strains. Each enzyme catalyzes a reaction yielding the substrate for the subsequent enzyme. Thus, each enzyme must be properly targeted and must function at high efficiency in its respective location in the secretory pathway. The application of a combinatorial library approach has been essential to generate Pichia pastoris strains harboring combinations of mannosidases, glycosyltransferases, GlcNAc/Gal transporters, and Gal epimerase (Li, H., Sethuraman, N., Stadheim, T. A., Zha, D., Prinz, B., Ballew, N., Bobrowicz, P., Choi, B. K., Cook, W. J., Cukan, M., Houston-Cummings, N. R., Davidson, R., Gong, B., Hamilton, S. R., Hoopes, J. P., Jiang, Y., Kim, N., Mansfield, R., Nett, J. H., Rios, S., Strawbridge, R., Wildt, S., Gerngross, T. U.: Optimization of humanized IgGs in glycoengineered Pichia pastoris. Nat. Biotechnol. 2006;24:210-215). These libraries consist of an array of catalytic domains in combination with an array of targeting sequences. The work leading up to the production of GlcNAcMan5 structures in Pichia pastoris has been published with full details of how to create and screen libraries of hundreds of fusions between leader sequences and catalytic domains from various species encoding for α-1,2 mannosidase, and N-acetylglucosaminyltransferase (GlcNAc transferase or GnT) I (Choi, B. K., P. Bobrowicz, R. C. Davidson, S. R. Hamilton, D. H. Kung, H. Li, R. G. Miele, J. H. Nett, W. S., and T. U. Gerngross, Use of combinatorial genetic libraries to humanize N-linked glycosylation in the yeast Pichia pastoris. Proc Natl Acad Sci USA, 2003,100(9):5022-5027). Further publications described generation of Pichia pastoris strains harboring combinations of α-1,2 mannosidase, N-acetylglucosaminyltransferases I and II, and mannosidase II (Hamilton, S. R., P. Bobrowicz, B. Bobrowicz, R. C. Davidson, H. Li, T. Mitchell, J. H. Nett, S. Rausch, T. A. Stadheim, H. Wischnewski, S. Wildt, and T. U. Gerngross, Production of complex human glycoproteins in yeast. Science, 2003,301(5637):1244-1246). The approach described herein and the resulting strains provide the basis for further engineering efforts of a complex human glycoprotein, such as lactoferrin (LF), in Pichia pastoris expression system.

Lactoferrin, an iron-binding glycoprotein, is considered a first line defense protein involved in protection against microbial infections (Lonnerdal, B., Iyer, S.: Lactoferrin: molecular structure and biological function. Annu. Rev. Nutr. 1995,15:93-110) and prevention of systemic inflammation (Baveye, S., Elass, E., Mazurier, J., Spik, G., Legrand, D.: Lactoferrin: a multifunctional glycoprotein involved in the modulation of the inflammatory process. Clin. Chem. Lab. Med. 1999,37:281-286). More recently, lactoferrin has been implicated in immunoregulatory functions (Kruzel, M. L., Zimecki, M.: Lactoferrin and immunologic dissonance: clinical implications. Arch. Immunol. Ther. Exp. 2002,50:399-410), as a modulator of vaccine function, and containing chemoprotective activity. There is evidence that lactoferrin has ability to reconstitute a drug-induced immune imbalance including cyclophosphamide or busulfan-induced immune suppression in mice (Artym J, Zimecki M, Kuryszko J, Kruzel M L. Lactoferrin accelerates reconstitution of the humoral and cellular immune response during chemotherapy-induced immunosuppression and bone marrow transplant in mice. Stem Cells Dev. 2005;14(5):548-55). The primary structure of human LF is characterized by a single polypeptide chain containing 692 amino acids organized in two highly homologous lobes, designated the N- and C-lobe, each capable of binding single ferric ion (Fe⁺⁺⁺). There are three possible N-linked glycosylation sites in hLF, one at Asn138, a second site at Asn479, and a third site at Asn624; differential utilization of these sites results in distinct glycosylation variants (Samyn-Petit, B., Wajda Dubos, J. P., Chirat, F., Coddeville, B., Demaizieres, G., Farrer, S., Slomianny, M. C., Theisen, M., Delannoy, P.: Comparative analysis of the site-specific Nglycosylation of human lactoferrin produced in maize and tobacco plants. Eur. J. Biochem. 2003,270:3235-3242). Human LF glycans are the N-acetyllactosaminic type, α1,3-fucosylated on the N-acetyl-glucosamine residue linked to the peptide chain. There are two primary forms of human lactoferrin, one contained in exocrine secretions and the other form is present in the secondary granules of neutrophils. While the two forms of human LF are identical in their amino acid sequence, they differ in sugar moieties. The granulocytic LF is not fucosylated thereby allowing transduction of signals that do not require fucose specific receptors such as the mannose receptor. In addition, while the secreted form is thought to be involved in the host defense against microbial infection at mucosal sites, the granulocytic lactoferrin has notable immunomodulatory function.

Human lactoferrin has been cloned and expressed in several heterologous systems including transgenic plants and animals. It appears however that full functionality is dependent upon proper glycosylation of the molecule. Currently there is no recombinant human LF that would be fully compatible with its natural counterpart. According to the present invention the glycoengineered Pichia pastoris is used to produce a highly uniform N-glycan structure with terminal galactose (Gal₂GlcNAc₂Man₃GlcNAc₂) N-linked to the protein chain of rhLF. Because sialylation, the final step of human glycosylation, is particularly difficult to accomplish in yeast, the in vitro process to create terminally sialylated N-glycan (Sia₂Gal₂GlcNAc₂Man₃GlcNAc₂) of rhLF is used. The lactoferrin of the present invention is the first fully humanized and biologically active lactoferrin ever described.

### SUMMARY OF THE INVENTION

The present invention relates to a composition of matter consisting of recombinant human lactoferrin (rhLF) expressed in the glycoengineered Pichia pastoris capable to furnish a mammalian type of N-glycan structure that is further modified by in vitro sialylation to accomplish a fully humanized recombinant product. In one embodiment the rhLF is expressed, isolated and purified from the glycoengineered Pichia pastoris and subjected to sialyltransferase in a presence of sialic acid to achieve a terminal sialylation of rhLF N-glycan structure. The present invention is further directed to the use of rhLF in humans. More specifically the present invention is directed to the use of rhLF for reconstitution of impaired immune responses, in particular, drug-induced immunosupression of cellular and/or humoral responses in humans. The present invention is also directed to the use of rhLF to prevent or treat a disease-induced immune impairment in humans.

### BRIEF DESCRIPTION OF DRAWINGS

FIG. 1 shows SDS-PAGE analysis of samples at different stage of purification: Coomassie blue stain (a) and Western blot with antibodies anti-hLF (b) and anti-HCP (c) of samples at different purification steps. Lane 1, broad range molecular weight standards; lane 2, rhLF fermentation supernatant; lane 3, SP Sepharose Fast Flow eluant; lane 4, Heparin Sepharose 6 Fast Flow eluant. Anti-HCP: antibody against Pichia host cell proteins;

FIG. 2 shows MALDI-TOF spectra analysis of non-sialylated (a) and sialylated (b) rhLFs. Protein samples were diluted 1:1 with sinapinic acid in 0.1% trifluoroacetic acid and 50% acetonitrile, then spotted on a MALDI plate and examined using positive mode on an Applied Biosystems Bioanalyzer MALDI-TOF;

FIG. 3 shows MALDI spectra analysis of glycan structures. Glycans were removed from rhLF with PNGase F and subjected to MALDI-TOF analysis in the positive ion mode for non-sialylated rhLF (a) and the negative ion mode for sialylated rhLF (b). The major peak observed in the non-sialylated rhLF N-glycan spectrum corresponds to Gal₂GlcNAc₂Man₃GlcNAc₂ (GS5.0) whereas the major peak in the sialylated rhLF N-glycan spectrum corresponds to Sia₂Gal₂GlcNAc₂Man₃GlcNAc₂ (GS6.0).J774A.1 (top) or RAW 264.7 cells (bottom) were stimulated with LPS (200 ng/ml) and increasing concentrations of Lactoferrin (1 to 1000 μg/ml). Supernatants were assessed for IL-12 (closed bar) and IL-10 (open bar). Average values (pg/ml) with standard errors are shown. *, p<0.05 compared to LPS alone;

FIG. 4 shows peptide mapping of non-sialylated rhLF. The dotted chromatogram displays the chromatographic UV trace for tryptic peptides from glycosylated rhLF; the solid line corresponds to the trace for deglycosylated tryptic peptides. Fractions from the glycosylated sample were then treated with PNGase F and run on liquid chromatography-coupled mass spectrometer via nanospray to identify peptide sequences. Fragments corresponding to deglycosylated and non-glycosylated Nglycosylation sites (N138, N479, N624) were identified in both samples and peaks containing these fractions were indicated with asterisks. Glycopeptides (N138 and N479) were identified in peaks marked with arrows;

FIG. 5 shows rhLF effect on reconstitution of the secondary humoral immune response in mice suppressed by methotrexate. Splenocytes isolated from sheep red blood cell (SRBC) primed mice were incubated with SRBC, alone in the presence of methotrexate (MTX). Antibody forming cells (AFC) were evaluated after 4 days. Cells were cultured in the presence of non-sialylated LF (LF A), sialylated LF (LF B), or milk derived LF (HLF Sigma). All LF concentrations were 1 μg/ml. The results are shown as mean values of AFC number from five wells±SE, calculated per 106 viable cells. Statistical analysis of groups: control vs. LFA NS; control vs. LF B NS; control vs. HLF Sigma p=0.0068; control vs. MTX p=0.0001; control vs. LF A+MTX p=0.0001; control vs. LF B+MTX NS; control vs. HLF Sigma+MTX NS; MTX vs. LF A+MTX NS; MTX vs. LF B+MTX p=0.0001; MTX vs. HLF Sigma+MTX p=0.0001 (ANOVA); and

FIG. 6 shows in vitro effect of rhLF in the secondary immune response. Splenocytes isolated from sheep red blood cell (SRBC) primed mice were incubated with SRBC, alone in the presence of methotrexate (MTX). Antibody forming cells (AFC) were evaluated after 4 days. Cells were cultured in the presence of sialylated LF (LF B). Free sialic acid was added alone, or in combination with LF (a). Alternatively, anti-sialoadhesin monoclonal antibody CD169 (sialoadhesin) was added before LF (1:250) (b). a Control vs. LF B p=0.0001; control vs MTX p=0.0001; control vs. Sia NS; control vs. LF B+Sia NS; control vs. LF B+MTX NS; control vs LF B+Sia+MTX p=0.0001; MTX vs. LF B+MTX p=0.0001; MTX vs LF B+Sia+MTX NS; LF B+MTX vs. LF B+Sia+MTX p=0.0023 (ANOVA). b Control vs. LF B p=0.0291; control vs. MTX p=0.0001; control vs. Ab NS; control vs. LF B+Ab+MTX p=0.0001; LF B vs. LF B+Ab p=0.0001; MTX vs. LF B+MTX p=0.0001; MTX vs. LF B+Ab+MTX NS; LF B+MTX vs. LF B+Ab+MTX p=0.0001 (ANOVA

### DESCRIPTION OF THE INVENTION

The present invention relates to a composition of matter consisting of recombinant human lactoferrin (rhLF) expressed in the glycoengineered Pichia pastoris capable to furnish a mammalian type of N-glycan structure that is further modified by in vitro sialylation to accomplish a fully humanized recombinant product. A more complete understanding can be obtained by reference to the following specific Examples also described in Glycoconjugate Journal (Choi, B-K, Actor, J K Rios, S, d'Anjou, M, Stadheim, T A, Warburton, S, Giaconne, E, Cukan, M, Li, H, Kull, A, Sharkey, N, Gollnick, P, Kocieba, M, Artym, J, Zimecki, M, Kruzel, M L, and Wildt, S. Recombinant human lactoferrin expressed in glycoengineered Pichia pastoris: Effect of terminal N-Acetylneuraminic acid on in vitro secondary humoral immune response. Glycoconjugate J. 2008,25:581-593). These Examples are described solely for the purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Strains, Culture Conditions and Reagents.

Escherichia coli strain TOP10 was used for recombinant DNA work. Pichia pastoris yAS309 (24) was used for generation of rhLF producing strains. Protein expression studies were done at room temperature in a 96-well plate format with buffered glycerol-complex medium (BMGY) consisting of 1% yeast extract, 2% peptone, 100 mM potassium phosphate buffer, pH 6.0, 1.34% yeast nitrogen base, 4×10-5% biotin, and 1% glycerol as a growth medium. The induction media were buffered methanol-complex medium (BMMY) consisting of 0.5% methanol and buffered dextrose-complex medium (BMDY) consisting of 2% dextrose, respectively. hLF standard purified from human milk was purchased from Sigma (St. Louis, Mo.). Restriction and modification enzymes were from New England BioLabs (Beverly, Mass.). All chemicals were ACS-grade, and CMP-sialic acid and trypsin were purchased from Sigma (St. Louis, Mo.), recombinant rat a 2,6-(N)-Sialyltransferase from Calbiochem (San Diego, Calif.), 2-aminobenzamide (2-AB) dye from Aldrich (St. Louis, Mo.). A polyclonal antibody against Pichia pastoris-derived host cell proteins (anti-HCP) was generated as follows; the culture supernatant from Pichia pastoris lacking hLF construct was purified in a similar fashion as described in the capturing step of rhLF. The purified proteins in PBS were used for polyclonal antibody generation in rabbits (Rockland Immunochemical Inc., Boyertown, Pa.).

### Expression Constructs and Generation of Production Strains.

For the signal sequence study, pPICZA (Invitrogen, Carlsbad, Calif.) was digested with EcoRI and KpnI, and the resulting pPICZA was ligated with 11 different signal sequences (EcoRI and blunt ended) and the codon-optimized hLF cDNA (blunt and KpnI ended) synthesized according to sequence disclosed in U.S. Pat. No. 6,066,469. For the promoter study, PpAOX1 promoter was replaced with the PpGAPDH promoter in pBK422 at BgIII and EcoRI sites. All expression constructs were sequence verified. For the generation of hLF production strains, PmeI-digested DNAs were transformed into yAS309 by electroporation, according to the Pichia pastoris expression kit handbook from Invitrogen. Table 1 registers designated plasmids and strains.

### Fermentation.

A seed culture was prepared by adding 1 ml of thawed cells to a 2 L baffled flask containing 400 ml of 4% BMGY medium. When an OD600 of 20±5 was reached, the seed culture was transferred to the production fermenter. The systems was controlled by Applikon 1030 Bio-controller with closed loop control of pH, temperature, dissolved oxygen concentration and foam control as described earlier (Li H, Sethuraman N, Stadheim T A, Zha D, Prinz B, Ballew N, Bobrowicz P, Choi B K, Cook W J, Cukan M, Houston-Cummings N R, Davidson R, Gong B, Hamilton S R, Hoopes J P, Jiang Y, Kim N, Mansfield R, Nett J H, Rios S, Strawbridge R, Wildt S, and Gerngross T U. Optimization of humanized IgGs in glycoengineered Pichia pastoris. Nat Biotechnol 2006,24:210-215). The pH was maintained at 6.0 throughout the fermentation. Fermentation runs were carried out in 15 L (12 L working) autoclavable glass bioreactors from Applikon (Foster City, Calif.).

### Protein Purification.

Primary clarification was performed at 4° C. for 15 min at 13,000×g by centrifugation in a Sorvall Evolution RC (Kendo lab products, Asheville, N.C.) followed by the microfiltration and diafiltration steps using a 0.1 ↑m cut-off 3600 cm² PES hollow fibre cartridge (CFP-1-E-8A) (GE Amersham, Pittsburgh, Pa.) and 5×0.1 m² Pellicon 2 Mini 10 kDa NMWCO regenerated cellulose ultrafiltration cassettes (A screen) (Millipore, Billerica, Mass.), respectively. Protease inhibitors pepstatin A and chymostatin (Sigma) were added to the supernatant after filtration steps in a concentration of 5 μg/ml and 3 μg/ml respectively. After the ultrafiltration-diafiltration-filtration steps, Pichia pastoris-derived rhLF was purified by two chromatographic steps; cation exchange chromatography using SP Sepharose Fast Flow followed by Sepharose 6 Fast Flow chromatography (GE Healthcare, Piscataway, N.J.). Briefly, SP Sepharose resin was equilibrated with 50 mM Tris-HCl, pH 8.0 while the supernatant media was adjusted at the same pH and conductivity around 5 mS/cm by the diafiltration procedure. The elution was done with 10 column volume (CV) of a gradient of 0-1 M NaCl in the same buffer. rhLF containing fractions were pooled and dialyzed against 50 mM Tris HCl, pH 7.5 overnight. As the final purification step the affinity resin, heparin Sepharose 6 Fast Flow (GE Healthcare), was used and the column was equilibrated with 50 mM Tris-HCl (pH 7.5). The pooled and dialyzed protein from SP Sepharose was loaded on Heparin Sepharose and washed in 3 steps. The first wash was done with 2 CV of the same buffer, followed by the second wash with 10 CV of a detergent buffer (10 mM CHAPS, 10 mM EDTA in 50 mM Tris-HCl, pH 7.5) to decrease endotoxin levels. The last wash was carried out with a 10 CV of 50 mM Tris-HCl (pH 7.5). The protein was eluted with a 10 CV of a gradient of 0-1 M NaCl. A fraction of the pooled protein was dialyzed against PBS (pH 7.2) and stored at 4° C. as a final product of non-sialylated hLF. The other fraction was dialyzed against 50 mM MES, pH 6.5 for the preparation of the in vitro sialylation. The in vitro sialylated rhLF was purified using heparin Sepharose 6 Fast Flow and dialyzed against PBS (pH 7.2), and stored at 4° C. Protein concentration was estimated using the method of Bradford as described (Bradford MM. A rapid and sensitive method for the quantitation of microgram quantities of protein utilizing the principle of protein-dye binding. Anal Biochem 1976,72: 248-254). Protein assay reagents were from Pierce Biotechnology (Rockford, Ill.). Bovine serum albumin (Pierce Biotechnology) and human milk lactoferrin (Sigma) were used as standards. Identification of rhLF is done by immunostaining with anti-human antibody. Proteins were separated by 4-20% gradient SDS-PAGE and then electroblotted onto nitrocellulose membrane (Schleicher & Schuell Inc., Keene, N.H.). The membrane was probed with rabbit anti-human LF antibody (anti-hLF) (1:1000) (Sigma, St. Louis, Mo.) and followed by goat anti-rabbit IgG antibody conjugated with horseradish peroxidase (1:4000) (Pierce, Rockford, Ill.). The results were visualized using an ImmunoPure Metal Enhanced DAB Substrate Kit (Pierce, Rockford, Ill.).

### Size Exclusion Chromatography and Reverse-Phase HPLC.

Proteins were separated with a BioRad BioSil SEC250 column (Hercules, Calif.) using a mobile phase of 100 mM sodium phosphate, pH 6.8, 150 mM NaCl and 0.05% sodium azide at 0.5 ml/min and detected at 280 nm. For the HPLC experiments proteins were separated with a Phenomenex Jupiter 5□ C4 300 Å column (Torrance, Calif.) using a 1.0 ml/min 39-min linear gradient from 95% to 30% buffer A (0.1% TFA in water). Buffer B was 0.08% TFA in acetonitrile. Temperature was maintained at 80° C. using a column oven. A Hitachi diode array detector monitoring at 280 nm was used for detection.

### MALDI-TOF Analysis of Glycans.

N-glycans were released and separated from hLF as described earlier (Choi B K, Bobrowicz P, Davidson R C, Hamilton S R, Kung D H, Li H, Miele R G, Nett J H, Wildt S, and Gerngross T U. Use of combinatorial genetic libraries to humanize N-linked glycosylation in the yeast Pichia pastoris. Proc Natl Acad Sci USA 2003,100: 5022-5027). Molecular weight was determined by using a Voyager linear matrix-assisted laser desorption/ionization time of flight (MALDI-TOF) mass spectrometer (Applied Biosystems) with delayed extraction. 2-aminobenzamide (2-AB) labeling was used to quantify N-glycan structures. A solution of 5% 2-AB dye and 6.3% sodium cyanoborohydride was prepared in 1:4 glacial acetic acid:DMSO. Five □l of this solution was added to dried glycan samples, mixed, and incubated for 2-3 h at 65° C. Each sample was applied to wells of a 96-well lysate plate (Promega Cat # A2241, Madison, Wis.) and then washed and pre-wetted with acetonitrile and adsorbed for 10-15 min; wells were then washed with 1 ml acetonitrile followed by three 1 ml 96% acetonitrile/4% water washes. Glycans were eluted three times with 0.4 ml water and dried in a centrifugal vacuum for 24 h. Labeled glycans were then separated by HPLC using a flow rate of 1.0 ml/min with a Prevail CHO ES 5-micron bead, amino-bound column using a 50-min linear gradient of 80% to 40% buffer A (100% acetonitrile). Buffer B consisted of 50 mM ammonium formate pH 4.4. Sialylated glycans were separated using a 30-min 80-40% Buffer A linear gradient with an additional 30-min gradient bringing buffer A from 40% to 0%. Labeled glycans were detected and quantified against standards using a fluorescence detector with an excitation of 330 nm and an emission at 420 nm.

### Trypsin Peptide Mapping.

2 mg of 0.25 mg/ml rhLF was diluted 1:2 with 10 M Guanidine HCl pH 7.8. To this mixture, dithiothreitol (DTT) was added to a final concentration of 10 mM and incubated at 37° C. for 1 h. Iodoacetic acid in Tris base neutralized with NH₄OH was added to a final concentration of 40 mM and incubated in the dark for 1 h. The sample was then buffer-exchanged and concentrated with a Vivaspin 50,000 MWCO concentrator to roughly 1 mg/ml in 100 mM Tris-HCl, pH 7.4. The sample was subsequently split into four aliquots with each receiving 2.6 □g of trypsin in 10 mM HCl. Samples were incubated overnight at 37° C. then boiled for 5 min. Two aliquots were deglycosylated with 5 □l of PNGase F for 30 min at 37° C. Peptides were resolved with a Phenomenex Jupiter 4 □ Proteo 90 Å column using an 87-min linear gradient of 95% to 55% Buffer A (0.1% TFA in water) at 1.0 ml/min. Buffer B was 0.08% TFA in acetonitrile. Temperature was maintained at 30° C. using a column oven. A Hitachi diode array detector monitoring at 220 nm was used for detection. 1 min fractions were collected from the column into a 96-well plate and dried.

### Nanospray Mass Spectrometry.

Digested peptides were dissolved in 50 μl 30% acetonitrile, 0.1% formic acid. Fractions with peptides that appeared on the HPLC trace after PNGase F digest (RT: 55 min, 56 min, 63 min, 64 min, 65 min, 66 min) were transferred to a 96-well Abgene sample plate and sealed with adhesive foil. A Thermo Finnigan LTQ mass spectrometer was set to data-dependent triple play mode to analyze the top 5 most intense peaks for 2.9 minutes through Advion Triversa Nanomate. Data were analyzed using SEQUEST (Thermo Finnigan, Waltham, Mass.).

In vitro Sialylation.

200 mU sialyltransferase and 3.875 mg CMP-Sialic acid (final concentration; 52.5 μM) was added to 31 mg of hLF in 50 mM MES pH 6.5, 10 mM MnCl₂, 0.73 μM chymostatin, and 52 ng/l pepstatin and incubated overnight at 25° C. Sialylated rhLF was dialyzed against 20 mM Tris (pH 8.0) overnight followed by purification using heparin Sepharose Fast Flow 6. The endotoxin levels were assayed using a limulus amebocyte lysate endochrome assay kit from Charles River Laboratories according to the manufacturer's instructions (Charleston, S.C.).

### Enzyme-Linked Immunosorbant Assay.

A high-protein binding 96-well plate (Costar) was coated with 100 μl/well of rabbit anti-human LF antibody (Sigma) diluted 1:5000 in PBS (EMD Biosciences, San Diego, Calif.) overnight at 4° C. Antibody was aspirated and the plate was blocked for 1 h at room temperature with 200 μl of 3% BSA in PBS. Blocking solution was aspirated and replaced with 100 μl of serial dilutions of commercially available human colostrum hLF (Sigma) and samples to be assayed. Standard hLF was diluted two-fold serially in PBS from 100 ng/ml to 0.1 ng/ml; fermenter samples were typically diluted 1:100 and then two-fold serially to 1:100,000. Standards and samples were incubated for 1 h at room temperature then aspirated and washed three times with 300 μl/well of 0.05% Tween 20 in PBS using a manifold plate washer. Wash buffer was aspirated and then 100 μl/well of HRP-conjugated anti-hLF (Jackson Immunoresearch, West Grove, Pa.) diluted 1:5000 in PBS was added and incubated for 1 h at room temperature. Wells were washed, 100 μl/well of 3,3′,5,5′-Tetramethylbenzidine (Sigma) was added and the final reaction terminated with 1 M H₂SO₄ after which absorbance at 450 nm was measured.

### Mice.

12-week old CBA male and female mice were used for the studies. All in vivo experiments were conducted under animal ethics committee approved guidelines.

### The Secondary Humoral Immune Response in Vitro.

Mice are primed with intraperitoneal administration of 0.2 ml 1% sheep red blood cells (SRBC) suspension. Splenocytes are isolated after four days and single cell suspensions were prepared in culture medium consisting of RPMI 1640, supplemented with 10% fetal calf serum, glutamine, sodium pyruvate, 2-mercaptoethanol and antibiotics. The cells are incubated in 24-well culture plates (5×10⁶ /ml/well) with 50 μl 0.005% SRBC. Lactoferrins are added to the cells cultures in concentration of 1 μg/ml at the initiation of culture, MTX at concentration of 0.25-0.5 mM, after 24 h, and sialic acid (0.5 mM) at 30 min before addition of LF. The antibody against mouse sialoadhesin (Serotec, rat anti-mouse CD169, clone 3D6.112, final dilution 1:250) is added to the cell cultures 1 h before LF. After four days the number of AFC was determined by the method of local hemolysis in agar (27). The results are shown as mean values of AFC number from 5 wells±SE, calculated per 10⁶ viable cells.

### Statistics

The differences across groups were determined by analysis of variance after testing homogeneity of variance by Levene's test. Individual grades were then compared using the Tukey's test for multiple comparisons. The data are expressed as: mean, mean ±SE (standard error) and mean ±SD (standard deviation). Differences were considered significant when p<0.05. The statistical analysis was performed using STATISTICA 6.0 for Windows.

Methods of molecular biology, immunology and biochemistry used but not explicitly described in this disclosure and these Examples are amply reported in the scientific literature and are well within the ability of those skilled in the art.

### EXAMPLE 1

Optimization of recombinant human lactoferrin expression in glycoengineered Pichia pastoris. The expression of rhLF is optimized by such factors as codon usage, signal sequence, promoter, pH, FeCl₃, and induction time. Previously, DNA codon optimization has successfully been applied to improve expression of heterologous proteins in yeast. In order to improve the translational efficiency of rhLF in Pichia pastoris, a codon-optimized nucleotide sequence encoding hLF is synthesized based on the original sequence published (Choi, B-K, Actor, J K Rios, S, d'Anjou, M, Stadheim, T A, Warburton, S, Giaconne, E, Cukan, M, Li, H, Kull, A, Sharkey, N, Gollnick, P, Kocieba, M, Artym, J, Zimecki, M, Kiruzel, M L, and Wildt, S. Recombinant human lactoferrin expressed in glycoengineered Pichia pastoris: Effect of terminal N-Acetylneuraminic acid on in vitro secondary humoral immune response. Glycoconjugate J. 2008,25:581-593). Amino acid codons are selected based on a Pichia pastoris codon usage table. A codon-optimized hLF cDNA is fused to eleven different signal sequences (pBK833˜pBK850 in Table 1) to identify the optimal secretion sequence facilitating translocation of rhLF into the secretory pathway and ultimately the culture medium. rhLF-producing strains representing eleven signal sequences were generated by transforming eleven expression constructs into yAS309. The efficiency of each signal sequence is evaluated by Western blot of the culture supernatants. Out of the signal sequences tested, S. cerevesiae alpha mating factor prepro (ScαMFppKR: pBK842) is selected in which ScαMFppKR is engineered to contain a Kex2p cleavage site (KR) at C-terminal of the signal sequence to facilitate processing of the protein prior to secretion of mature rhLF. The resulting strain is designated BK422. BK422 is further optimized to maximize rhLF production. The pH of medium can have an impact on the overall production of the protein of interest by increasing the activity of specific proteases secreted from the host strain as well as influencing protein stability. In order to minimize proteolysis and to enhance protein stability, protein production was tested at different pH values in the induction medium (BMMY) ranging from 6.0˜7.5. The pH of growth medium (BMGY) is maintained at 6.0. At pH over 6.5, proteolytic degradation of rhLF was observed. The optimal pH of the induction medium was found to be 6.0. No iron (FeCl₃) supplementation has been required.

Two different Pichia pastoris (Pp) promoters were evaluated for their ability to drive rhLF expression. An inducible promoter from Pichia pastoris: alcohol oxidase 1 (PpAOX1) and one constitutive promoter derived from the glyceraldehyde-3-phosphate dehydrogenase gene of Pichia pastoris (PpGAPDH) were evaluated for their ability to drive hLF expression. The AOX1 promoter is tightly regulated at the transcription level, that is, it is repressed in the presence of glucose whereas it is strongly induced in the methanol medium. rhLF was induced at 0.5% methanol under AOX1 promoter and at 2% dextrose under GAPDH promoter. The proteolytic degradation of rhLF was more pronounced under GAPDH promoter (BK427) than AOX1 promoter-driven protein expression (BK422). The AOX1 promoter-driven expression displayed the least amount of proteolysis and it was used to produce rhLF in a bioreactor. A time course study was performed to determine the optimal induction time. Methanol was added to the culture every 20 h to maintain rhLF induction during 4 day-fermentation. Samples were taken at different time-points during fermentation. Over the course of 4 days of induction, product yield continued to increase. However, an increased amount of proteolysis was also observed. An induction time of about 2-days was determined as an optimum.

### EXAMPLE 2

Production of recombinant lactoferrin in a bioreactor. rhLF production run is carried out with BK422 in a 15 L bioreactor described in materials and methods. rhLF is induced for 38 h and the product yield was 99.8 mg/L as measured by ELISA using hLF standard (Sigma). Proteolytic degradation is further reduced by supplementing the fermentation media with the protease inhibitors pepstatin A and chymostatin during induction. A total 841 mg rhLF is subjected to microfiltration followed by ultrafiltration/diafiltration steps as described earlier. After a series of filtration steps, the product recovery is 183 mg of rhLF (21% recovery).

### EXAMPLE 3

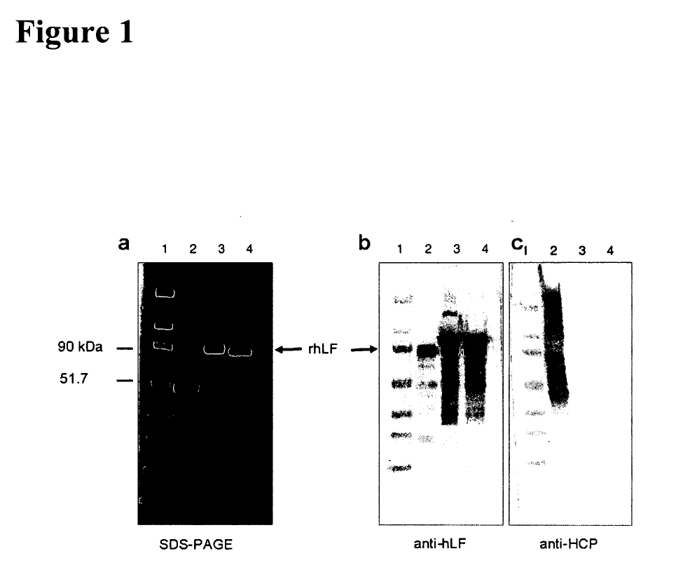

Purification of recombinant lactoferrin. rhLF is purified using SP Sepharose Fast Flow and Heparine Sepharose 6 Fast Flow. SP Sepharose FF captured rhLF effectively and is easily scalable from 300 μl to 160 ml of resin. Heparine Sepharose 6 FF is able to separate isoforms of hLF and chosen for the final purification step to further improve the protein purity. FIG. 1 shows the SDS-PAGE and Western blot analysis of rhLF purified from the fermentation supernatant. rhLF is successfully purified by SP Sepharose (FIGS. 1a, b and c). Western blot analysis using an anti-HCP antibody demonstrated that SP Sepharose Fast Flow is a good capturing step to obtain a highly pure protein free from host cell contaminants (FIG. 1c). As an advantage, passage over Heparin significantly reduced endotoxin levels. The combination of SP Sepharose and Heparin Sepharose enabled greater than 95% purification of rhLF with no isoforms observed as seen with the commercial standard. Analytical data support that the purified rhLF should be sufficient and acceptable as a homogenous molecule to proceed with defined in vivo testing.

### EXAMPLE 4

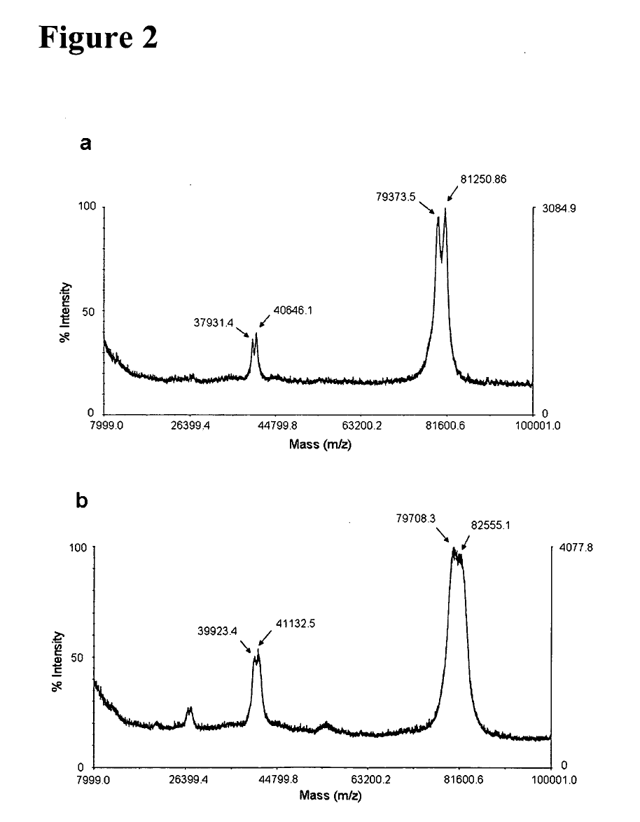

Generation of sialylated bi-anntenary rhLF by in vitro sialylation. In vitro sialylation of grhLF is carried out as described in earlier section “In vitro sialylation”, with purity reconfirmed by SDS-PAGE of the non-sialylated and sialylated rhLF. The protein N-terminal sequence is directly compared, indicating that the in vitro sialylation process is not affect the integrity of the N-terminus of the protein. The endotoxin levels are measured and determined to be 4 EU/mg (sialylated) and 3 EU/mg (non-sialylated), respectively. The purity of sialylated and non-sialylated rhLF samples is confirmed by reverse phase HPLC (data not shown). Size exclusion chromatography is also performed, demonstrating similarity in molecular weights of both molecules. Prominent peaks at m/z of 79373.5 and 81250.86 are observed by MALDI-TOF (FIG. 2a). A second pair of peaks at 39731.4 and 40646.1 suggests doubly-charged species of 79.3 and 81.2 kDa respectively, correlating with the masses of doubly- and triply-glycosylated rhLF (79.6 and 81.2 kDa, respectively). MALDI-TOF analysis of sialylated rhLF (FIG. 2b) showed a broader peak at about 81,000 m/z with the doubly charged spectra having a m/z of 40,000. The greater m/z ratio is most likely due to the presence of sialic acid in this sample.

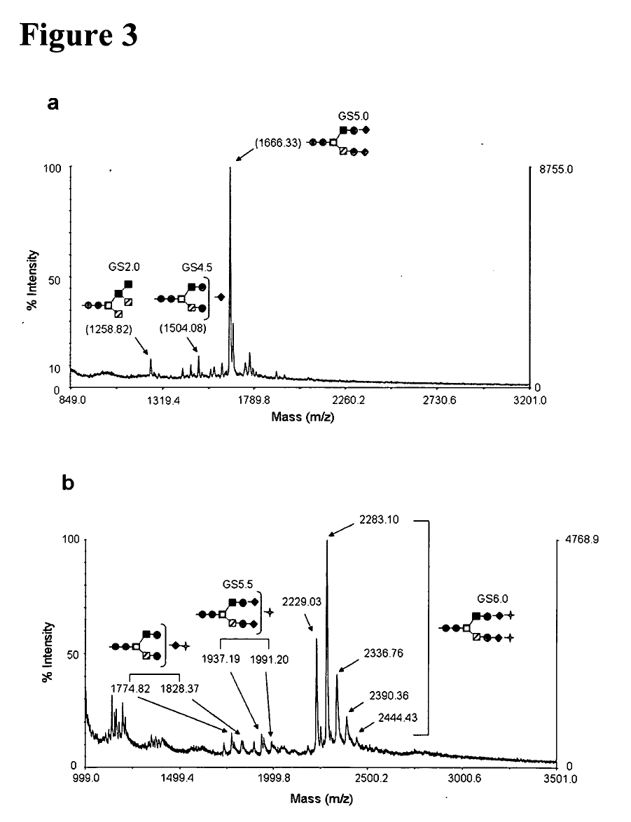

MALDI-TOF analysis is used to assess the N-linked glycan profile after enzymatic release from rhLF. Analysis of N-linked glycans released from non-sialylated rhLF in the positive ion mode showed a predominant mass of 1666.33 indicative of an afucosylated biantennary complex glycan with terminal galactose or Gal₂GlcNAc₂Man₃GlcNAc₂ (FIG. 3a). Additionally, the human glycoforms GalGlcNAc₂Man₃GlcNAc₂ (1504.08) and Man₅GlcNAc₂ (1258.82) are also observed, although less prominent. N-glycan of sialylated rhLF is analyzed by MALDI-TOF in the negative ion mode and showed a dominant cluster of ions in the range of 2229.03-2444.43 consistent with a mass describing Sia₂Gal₂GlcNAc₂Man₃GlcNAc₂ and associated cation adducts (FIG. 3b) Since MALDI-TOF provides a qualitative/semi-quantitative assessment of N-glycosylation, we employed a normal phase-HPLC analysis to quantify the relative abundance of specific N-linked glycan structures. Non-sialylated rhLF is determined to contain 50.99% Gal₂GlcNAc₂Man₃GlcNAc₂ (GS5.0) with other human-type glycans comprising GlcNAc₂Man₃GlcNAc₂ (GS4.0), GalGlcNAc₂Man₃GlcNAc₂ (GS4.5), GalGlcNAcMan₅ and GlcNAcMan₅ hybrids, and Man₅GlcNAc₂ (GS2.0). N-glycosylation analysis of the sialylated rhLF sample contained 46.2% terminally bisialylated N-linked glycans and 15.9% monosialylated N-linked glycans where most of Gal₁GlcNAc₂Man₃GlcNAc₂ (GS4.5) and Gal₂GlcNAc₂Man₃GlcNAc₂ (GS5.0) glycans are converted to SiaGal₂GlcNAc₂Man₃GlcNAc₂ (GS5.5) and Sia₂Gal₂GlcNAc₂Man₃GlcNAc₂ (GS6.0). However, GlcNAc₂Man₃GlcNAc₂ (GS4.0) and high mannose containing structures content from sialylated rhLF remained similar to non-sialylated rhLF because they are not the substrates for rat a 2,6-(N)-sialyltransferase.

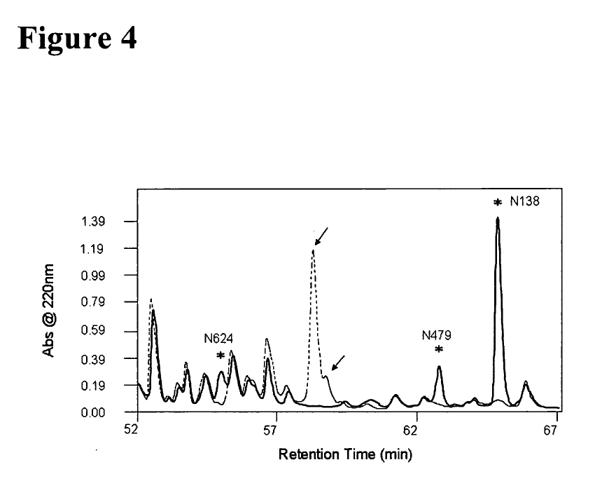

It has been described that glycosylation of hLF derived from mammalian cells occurs predominantly at two sites (N138 and N479) in approximately 85% of all hLF molecules. Glycosylation at a single site (N479) or at all three sites (N138, N479, and N624) occurs in only approximately 5% and 9% of hLF, respectively (van Berkel P H, van Veen H A, Geerts M E, de Boer H A, and Nuijens J H. Heterogeneity in utilization of N-glycosylation sites Asn624 and Asn138 in human lactoferrin: a study with glycosylation-site mutants. Biochem J 1996,319(Ptl):117-122). In order to determine N-glycosylation occupancy of each site, rhLF is digested with trypsin. One half of the sample is deglycosylated with PNGase F with the other one-half remaining untreated. Both samples are subjected to HPLC analysis. Whole chromatograms for each sample are overlaid in order to identify glycopeptides and the same peptides lacking glycan. FIG. 4 shows that the N-glycan occupancy was identified on chromatogram between 52 min and 67 min of retention time. To confirm the peptide sequences, HPLC fractions are collected and subjected to MS/MS analysis. For fractions from the untreated sample, PNGase F treatment is performed prior to MS/MS analysis in order to elucidate the glycopeptide sequences. The mixture of glycopeptides (N138 and N479) is identified, but the glycopeptide containing N629 was not resolved well. It is likely that the N629 glycopeptide's signal was buried because of co-elution with other peptides. However, based on the peptide mapping, it is confirmed that all three N-linked sites of rhLF are predominantly occupied with N-glycans whereas it has been shown that only 9% of hLF was N-glycosylated at all three sites if expressed in mammalian cell-derived systems.

### EXAMPLE 5

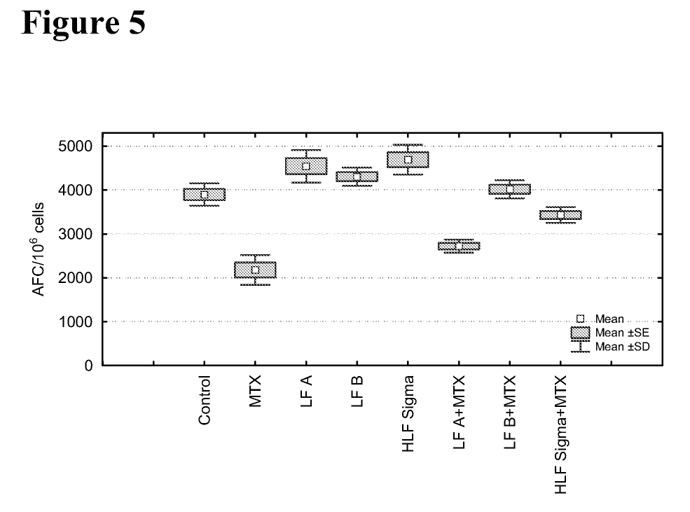

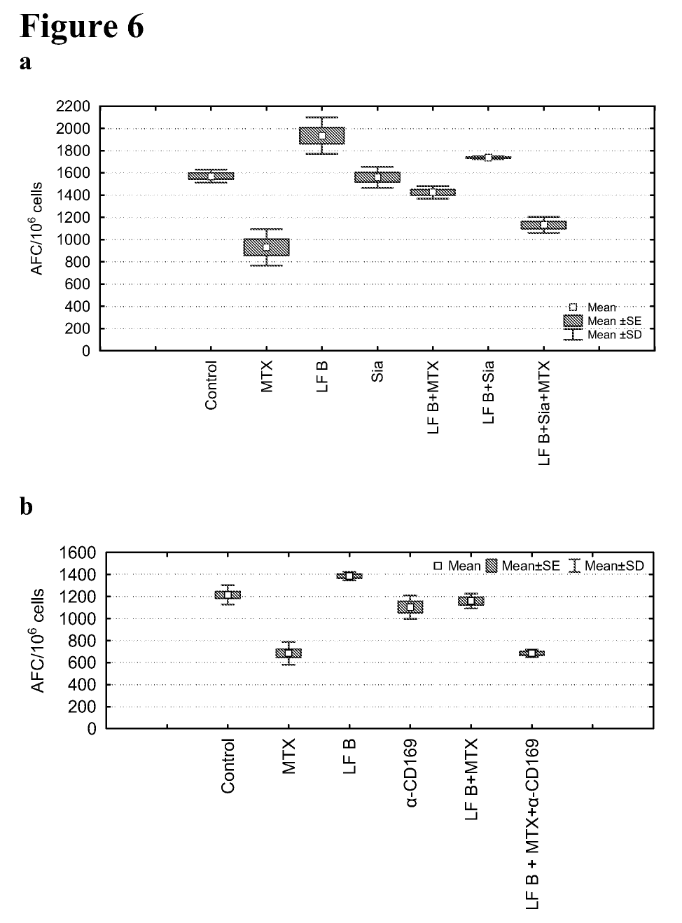

Reconstituting action of recombinant lactoferrin in the secondary humoral immune response in mice suppressed by methotrexate. It was previously demonstrated that bovine lactoferrin (bLF) can reduce methotrexate (MTX)-induced suppression of secondary humoral immune responses in vitro to sheep erythrocytes (SRBC) (Artym J, Zimecki M, and Kiruzel M L. Effect of lactoferrin on the methotrexate-induced suppression of the cellular and humoral immune response in mice. Anticancer Res 2004,24:3831-3836). Here both sialylated and non-sialylated rhLF are tested in vitro to demonstrate effects on the secondary humoral immune response. Human, milk-derived LF is used as a reference protein. The results (FIG. 5) demonstrated that sialylated rhLF is able to restore the number of antibody-forming cells in cultures treated with MTX. The non-sialylated rhLF is ineffective in the reduction of MTX-induced suppression of the secondary humoral immune response. Therefore it is clear that terminal capping of glycoengineered Pichia pastoris-derived rhLF with N-acetylneuraminic acid (sialic acid), by in vitro sialylation, become a critical step to produce fully humanized molecule that is capable to elicit proper immune responses. Since the results indirectly indicate the importance of sialic acid in mediation of the biological activity, a moderate concentration of free sialic acid is added to the cell cultures to block rhLF interaction with receptor (FIG. 6a). The results demonstrate that the addition of sialic acid significantly reduced the ability of sialylated rhLF to restore the MTX-mediated suppression. In addition, when monoclonal antibody to CD169, directed against the murine sialoadhesin, is added to the culture, the sialylated rhLF is unable to reverse the MTX-mediated suppression (FIG. 6b). This demonstrates a requirement of rhLF to bind to sialoadhesin for functional activity. In addition it is concluded that because both the protein and sugar moiety of rhLF are compatible with their natural counterparts, the composition of present invention shows no concern of immunogenicity when administered into humans. More importantly it is demonstrated for the first time that proper glycosylation of rhLF is required to effectively reconstitute drug-induced immunosupression.

### Use of Composition

According to the present invention, rhLF derived from the glycoengineered Pichia pastoris yAS309 and in vitro sialylated is further formulated into any pharmaceutically acceptable carrier for administration in the form of powder, aqueous or non-aqueous solution in humans. Preferably rhLF of present invention is delivered systemically (parenterally) in any pharmaceutically acceptable carrier that will be readily apparent to the skilled artisan.

According to the present invention the rhLF is preferably present in the formulation at a level of 0.01 milligram to 100 milligram, more preferably between 0.1 to 20 milligram, based on 1 milliliter or 1 gram of the carrier. An effective amount of rhLF varies depending on the individual treated and the form of administration. Preferable in treating individual, a single dose of 0.01 milligram to 100 milligrams, more preferable 0.1 milligram to 10 milligram of rhLF per kilogram of body weight is administrated parenterally (intravenously). rhLF can also be delivered orally or as a liposomal formulation, including transdermal patches.

According to the present invention, rhLF can be incorporated as an adjuvant composition in formulation with any vaccine or other adjunct therapeutic agents to protect against immunological imbalance or restore a disease and/or drug-induced immune impairment. Preferably rhLF is incorporated as an adjuvant composition in formulation with any vaccine such as a) vaccines containing killed microorganisms—examples are vaccines against flu, cholera, bubonic plague, and hepatitis A; b) vaccines containing live, attenuated microorganisms—examples include yellow fever, measles, rubella, Bacille Calmette-Guérin (BCG) and mumps; c) toxoids—examples of toxoid-based vaccines include tetanus and diphtheria; d) subunit—examples include the subunit vaccine against human papillomavirus (HPV) that is composed of the viral major capsid protein; e) prophylactic or therapeutic cancer—examples include antigen BCG for prostate cancer treatment, or HPV antigens for cervical cancer; f) allergen vaccines—example include ragweed pollen vaccine; g) autoimmune vaccine—example include use of antigens to induce tolerance against self antigens, for example autoimmune responses leading to induction of myasthenia gravis or other autoimmune reactions.

Furthermore, according to the present invention rhLF is incorporated as a therapeutic agent against progression of systemic inflammatory response syndrome (SIRS) into sepsis as described in U.S. Pat. No. 6,613,741. In particular, rhLF of the present invention is used to combat the methicillin-resistant Staphylococcus aureus (MRSA) infection, which is a major cause of sepsis in patients who are disease or drug-immunosuppressed. rhLF of the present invention is also used for prevention and/or treatment of any drug resistant infections.

In addition, rhLF can be incorporated as an adjunct composition in formulation alone or with any other therapeutic agent to modulate the immune responses during development of the age-related and chronic disorders, including neurodegenerative and immune hypersensitivity disorders in humans. In particular, lactoferrin is used to alleviate immune dissonance by assisting in the development of T-helper cell polarization during the insult-induced oxidative stress in humans.

Also, according to the present invention rhLF is used to modulate the Th1/Th2 balance in the context of immune homeostasis. Although, many pathological phenomena have been correlated with ROS, the role of oxidative stress in such chronic disorder-related decline or increase of T-cell activity is not yet clear. Still, according to the present invention lactoferrin is used to counterbalance allergen-reactive Th2 responses, also known as type 1 hypersensitivity (immediate) including allergy.

It is understood that the examples and embodiments described herein are for illustrative purposes only and that various modifications or changes in light thereof will be suggested to persons skilled in the art and are to be included within the spirit and purview of this application and scope of the appended claims.

TABLE 1
Strains and plasmids used in this study

| Strain and | | |
|---|---|---|
| plasmid | Description | Source |
| TOP10 | E. coli (F-mcrA Δ(mrr-hsdRMS-mcrBC) | Invitrogen |
| | φ80lacZΔM15 ΔlacX74 recA1 araD139 | (Carlsbad, |
| | Δ(ara-leu)7697 galU galK rpsL (StrR) | CA) |
| | endA1 nupG) | |
| yAS309 | Glyco-engineered Pichia pastoris | H. Li et al. |
| | | (24) |
| BK422 | pBK842 in yAS309 | This study |
| BK427 | pBK850 in yAS309 | This study |
| pPICZA* | PpAOX1 promoter, Zeo^{R} | Invitrogen |
| pGAPZA* | PpGAPDH promoter, Zeo^{R} | Invitrogen |
| pBK833 | S. cerevisiae α-mating factor pre + hLF in | This study |
| | pPICZA | |
| pBK834 | α-amylase signal sequence (ss) + hLF in | This study |
| | pPICZA | |
| pBK835 | Glucoamylase ss + hLF in pPICZA | This study |
| pBK836 | Human serum albumin ss + hLF in pPICZA | This study |
| pBK837 | Inulinase ss + hLF in pPICZA | This study |
| pBK838 | Invertase ss + hLF in pPICZA | This study |
| pBK839 | P. pastoris KAR2 ss + hLF in pPICZA | This study |
| pBK840 | S. cerevisiae killer toxin 1 ss + hLF in pPICZA | This study |
| pBK841 | P. pastoris phosphotase 1 ss + hLF in | This study |
| | pPICZA | |
| pBK842 | S. cerevisiae α-mating factor prepro + hLF in | This study |
| | pPICZA | |
| pBK843 | Chicken lysozyme ss + hLF in pPICZA | This study |
| pBK850 | S. cerevisiae α-mating factor preproKR + hLF | This study |
| | in pGAPZA | |

*Identical signal sequence used with different promoter sequences.

What is claimed:

1 A composition consisting of isolated recombinant human lactoferrin (rhLF) wherein the human DNA clone-expressed lactoferrin is glycosylated and sialylated to have a mammalian type of N-glycan structure (Sia₂Gal₂GlcNAc₂Man₃GlcNAc₂).

2. The composition of claim 1 wherein said human DNA clone-expressed lactoferrin is expressed by a glycoengineered Pichia pastoris.

3. The composition of claim 1 wherein said human DNA clone-expressed lactoferrin is expressed by a glycoengineered Pichia pastoris and said lactoferrin is further in vitro sialylated.

4. The composition of claim 1 wherein said composition elicits immune responses with respect to disease or drug-induced immune impairment in humans.

5. A pharmaceutical composition according to claim 1 wherein the composition is used to elicit immune responses with respect to disease or drug-induced immune impairment in humans.
